(19) United States
(12) Patent Application Publication
(10) Pub. No.: US 2008/0063668 A1
(43) Pub. Date: Mar. 13, 2008

(54) Nitroimidazole and terconazole composition and method

(76) Inventors: Robert J. Borgman, Mundelein, IL (US); James E. Juul, Wauconda, IL (US)

Correspondence Address: Olson & Cepuritis, LTD., 20 NORTH WACKER DRIVE, 36TH FLOOR, CHICAGO, IL, 60606, US

(21) Appl. No.: 11/519,296

(22) Filed: Sep. 12, 2006

(51) Int. Cl. A61K 31/4196; A61K 9/00

(52) U.S. Cl. 424/400; 514/383

(57) ABSTRACT

An aqueous tinidazole and terconazole composition comprises at least about 0.5 percent by weight of tinidazole and about 0.4 to about 3 percent by weight of terconazole dissolved in water, and a crystallization-inhibiting amount of an organic acid. Preferably the organic acid is citric acid. The composition is free from crystals at an ambient temperature of about 20° C. Methods of preparing the composition are also described.

### FIELD OF THE INVENTION

The invention relates generally to compositions comprising terconazole and a nitroimidazole, which are useful for treatment of microbiological infections. More particularly, the invention relates to aqueous compositions comprising terconazole and tinidazole and methods of preparing said compositions.

### BACKGROUND OF THE INVENTION

Tinidazole (1-[2-(ethylsulfonyl)ethyl]-2-methyl-5-nitroimidazole) (C₈H,₃N₃O₄S) is a synthetic nitroimidazole antibacterial and antiprotozoal agent which has been useful for treatment of various infections including bacterial vaginosis (BV) and vaginal trichomoniasis as an oral composition containing about 500 mg of tinidazole. Tinidazole exhibits activity against Trichomonas vaginalis, Giardia duodenalis, and Entamoeba histolytica protozoa. Tinidazole is also active against most anaerobic bacteria, including bacterial vaginosis pathogens such as Bacteriodes species, Peptostreptococcus species, and other anaerobes. The in vitro activity of tinidazole against other BV-associated bacteria such as Mobiluncus species, Gardnerella vaginalis, and Atopobium vaginae is limited, however.

Tinidazole has a solubility in water of about 0.45 percent by weight at neutral pH. It is difficult to obtain stable, physiological-acceptable aqueous tinidazole solutions at concentrations above about 0.45 percent by weight, which are free from tinidazole crystals.

Terconazole (cis-1-[p[[2-(2,4-dichlorophenyl)-2-( 1 H-1,2,4-triazol-1-ylmethyl)-1,3-dioxolan-4-yl]methoxy]phenyl]-4-isopropylpiperazine) (C₂₆H₃₁C₁₂N₅O₃) is a drug which has been useful for treatment of vaginal yeast infections in a cream composition containing about 0.4 or 0.8 percent by weight terconazole.

Terconazole is substantially insoluble in water at neutral pH. Terconazole hydrochloride has a solubility of only about 0.007 percent by weight in water at neutral pH. It is difficult to obtain terconazole solutions in water at concentrations required for therapeutic effect, and which are free from terconazole crystals.

There is an ongoing need for aqueous solutions that include at least about 0.5 percent by weight dissolved tinidazole and compositions containing about 0.4 to about 3 percent by weight dissolved terconazole, which are free from crystals. The present invention provides aqueous compositions containing both soluble tinidazole and soluble terconazole in a single composition in therapeutically effective amounts.

### SUMMARY OF THE INVENTION

An aqueous composition of the invention comprises at least about 0.5 percent by weight of tinidazole and about 0.4 to about 3 percent by weight of terconazole dissolved in water, and a crystallization-inhibiting amount of an organic acid. The composition is free from terconazole and tinidazole crystals at an ambient temperature of about 20° C. Preferably the organic acid is a water-soluble polybasic organic acid (e.g., citric acid).

In a preferred embodiment, the solution comprises up to 0.6 percent by weight of tinidazole and at least about 0.8 percent by weight of terconazole dissolved in water. In some preferred embodiments, terconazole can be present in the composition up to about 3 percent by weight.

In some preferred embodiments, the organic acid is present in the composition in a molar amount at least equal to the molar amount of tinidazole dissolved in the solution. In another preferred embodiment, the organic acid is present in the composition in a molar amount of at least equal to the total molar amount of tinidazole and terconazole dissolved in the solution. Preferably, the organic acid is present in the composition at a concentration in the range of about 50 to about 150 mole percent based on the combined moles of tinidazole and terconazole dissolved in the composition. Citric acid is a particularly preferred organic acid.

A method aspect of the present invention involves preparing an aqueous solution comprising at least about 0.5 percent by weight of tinidazole and about 0.4 to about 3 percent by weight of terconazole dissolved in water. The method comprises dissolving tinidazole and terconazole in water containing a crystallization-inhibiting amount of an organic acid.

In addition to an increase in solubility of tinidazole when combined with terconazole, the resulting combination also exhibited dual-drug cooperation in inhibiting the growth of various bacterial organisms.

### DETAILED DESCRIPTION OF PREFERRED EMBODIMENTS

The terms “pharmaceutically acceptable”, “physiologically tolerable”, “physiologically compatible”, and grammatical variations thereof, as used herein and in the appended claims as they refer to electrolytes (e.g., salts), bases, diluents, preservatives, buffers and other excipients, are used interchangeably and represent that the materials are capable of topical administration to human skin and to the human vagina without the production of medically unacceptable levels of undesirable physiological effects such as irritation, itching, stinging, or systemic effects such as nausea, dizziness, and the like.

The present invention provides aqueous compositions having a concentration of dissolved tinidazole of at least about 0.5 percent by weight and a concentration of dissolved terconazole in the range of about 0.4 to about 3 percent by weight, which are free from tinidazole and terconazole crystals. Concentrations of soluble tinidazole in the compositions of the invention can be as high as about 0.6 percent by weight. In the compositions of the present invention, tinidazole and terconazole are solubilized by the presence of a crystallization-inhibiting amount of an organic acid.

Suitable organic acids for use in the composition of the present invention include without limitation lower alkyl (i.e., C₁ to C₄) carboxylic acids such as acetic acid, and polybasic acids such as citric acid, tartaric acid, malic acid, polyacrylic acids, and the like. In some preferred embodiments the organic acid is present in the composition in a molar amount of at least about equal to the combined molar amount of tinidazole and terconazole dissolved in the solution. Preferably, the organic acid is present in the composition at a concentration in the range of about 50 to about 150 mole percent based on the combined moles of tinidazole and terconazole dissolved in the composition.

The compositions of the present invention optionally can include a physiologically tolerable preservative, as well as pharmaceutically acceptable excipients, thickening agents, and the like, so long as the optional components do not interfere with the solubility of the tinidazole or terconazole in the aqueous phase.

Suitable physiologically tolerable preservatives include bacteriostats, preservatives, inhibitors, and the like, such as methyl, ethyl, propyl, and butyl esters of parahydroxybenzoic acid (parabens); propyl gallate; sorbic acid and its sodium and potassium salts; propionic acid and its calcium and sodium salts; 6-acetoxy-2,4-dimethyl-m-dioxane; 2-bromo-2-nitropropane-1,3-diol; salicylanilides such as dibromosalicylanilide and tribromosalicylamilide, the cis isomer of 1-(3-chloroallyl)-3,5,7-triaza-1-azanidadamantane chloride; hexachlorophene; sodium benzoate; chelating agents such as ethylene diaminetetraacetic acid (EDTA), citric acid, and their alkali metal salts; phenolic compounds such as butyl hydroxyanisole, butyl hydroxytoluene, chloro- and bromo-cresols, and the like; quaternary ammonium compounds such as benzalkonium chloride; aromatic alcohols such as 2-phenylethyl alcohol and benzyl alcohol; chlorobutanol; quinoline derivatives such as iodochlorohydroxyquinoline; and the like.

Pharmaceutically acceptable excipients that can be included in the compositions of the present invention include, for example, physiologically tolerable thickeners, surfactants, colorants, fragrances, water-soluble or water-miscible co-solvents, and the like, which are well known in the art. The compositions of the invention can be free-flowing solutions or gels.

Gelled compositions include a thickening agent, such as a hydroxypropyl methylcellulose (hypromellose) compound, a crosslinked polyacrylic acid, and the like. Examples of suitable crosslinked polyacrylic acids include CARBOPOL® thickening agents, and NOVEON® brand polycarbophil crosslinked polyacrylic acids, available from Noveon, Inc., Cleveland Ohio. A combination of two or more thickening agents can be utilized, as well. Crosslinked polyacrylic acids can also act as the organic acid to solubilize the terconazole and nitroimidazole.

The present invention also provides a method for preparing a solution comprising at least about 0.5 percent by weight of tinidazole and about 0.4 to about 3 percent by weight of terconazole dissolved in water, free from crystals at an ambient temperature of about 20° C. The method comprises dissolving tinidazole and terconazole in an aqueous solution containing a crystallization-inhibiting amount of an organic acid as described hereinabove.

Preferably, an amount of tinidazole is dissolved in the solution to obtain a tinidazole concentration of about 0.5 to about 0.65 percent by weight. An amount of terconazole is dissolved in solution to obtain a terconazole concentration in the range of about 0.4 to about 3 percent by weight, preferably at least about 0.8 percent by weight. The concentration of organic acid in the solution preferably is at least about equal to the total molar amount of tinidazole and terconazole dissolved in the solution.

Another aspect of the present invention is an article of manufacture comprising packaging material and at least one composition of the invention in at least one sealed container within the packaging material. Preferably, the compositions are gels containing a thickening agent. The container comprises a label that includes printed indicia describing the contents, such as a listing of ingredients, the manufacturer's name and address, and the like. Preferably, the packaging material also includes a printed insert including detailed information on the composition, its method of administration for treatment of infections, side effects, contraindications, and the like indicia, which may be required by governmental agencies responsible for regulation of pharmaceutical products. The articles of manufacture may also include applicators, such as a tubular applicator that can be used in conjunction with a storage vessel or a squeezable tube to aid in applying the compositions of the invention (e.g., into the vagina). In addition, the container can be a single use packet or a pre-filled applicator.

The following non-limiting examples further illustrate the present invention.

### EXAMPLE 1

### Solubilization of Tinidazole in Water

Weighed amounts of tinidazole were added to separate containers including an acid in water, applying heat, if necessary, to dissolve the tinidazole. The amount of acid was selected to be about equimolar with the amount of tinidazole added. After the tinidazole was dissolved, each solution was cooled to about 2° C. to force crystallization of some of the tinidazole. If crystals were not observed upon cooling, seed crystals of tinidazole were added. After crystal formation was observed, the solutions were allowed to warm to room temperature (about 20° C.). Each solution was observed at about 20° C. over a period. of at least 14 days to determine if the crystals would re-dissolve. The absence of crystals after 14 days indicates effective dissolution of tinidazole. Table 1 includes dissolution data for a number of compositions comprising varying amounts of tinidazole and various acids. The data in Table 1 clearly indicate that solutions having a tinidazole concentration of at least about 0.5 percent by weight and free from tinidazole crystals at 20° C. were obtained utilizing citric acid, hydrochloric acid, acetic acid, tartaric acid, and malic acid. Tinidazole concentrations as high as about 0.55 percent by weight were obtained utilizing tartaric acid and citric acid.

TABLE 1

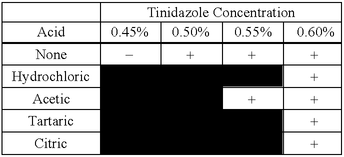

− means no crystals present
+ means crystal are present

### EXAMPLE 2

### Solubilization of Terconazole in Water

Weighed amounts of terconazole were added to separate containers including an acid in water, applying heat, if necessary, to dissolve the terconazole. The amount of acid was selected to be about equimolar with the amount of terconazole added. After the terconazole was dissolved, each solution was cooled to about 4° C. to force crystallization of the terconazole. If crystals were not observed upon cooling, seed crystals of terconazole were then added and the solutions were again cooled to about 4° C. for about 72 hours. After allowing for crystal formation in all solutions, the solutions were allowed to warm to room temperature (about 20° C.). Each solution was observed at about 20° C. over a period of about 14 days to determine if the crystals would re-dissolve. The absence of crystals after 14 days indicates that terconazole was soluble in the composition at the prepared concentration.

Table 2 includes dissolution data for a number of compositions comprising varying amounts of terconazole and various acids. The data in Table 2 clearly indicate that solutions having a terconazole concentration of at least about 3 percent by weight and free from terconazole crystals at 20° C. were obtained utilizing tartaric acid and malic acid. In a separate study, citric acid was found to solubilize terconazole at 2.4 percent by weight.

TABLE 2

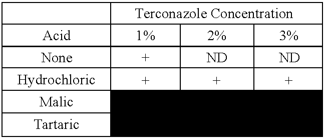

− means no crystals present
+ means crystal are present
ND means experiment not performed

### EXAMPLE 3

### Combined Solubilization of Tinidazole and Terconazole in Water

Weighed amounts of tinidazole and terconazole were added to separate containers including citric acid in water, applying heat, if necessary, to dissolve the tinidazole and terconazole. The amount of citric acid was selected to be about equimolar with the molar amount of tinidazole and terconazole added. After the tinidazole and terconazole was dissolved, each solution was cooled to about 4° C. to force crystallization of the tinidazole and terconazole. If crystals were not observed upon cooling, seed crystals of tinidazole were added and the solutions were again cooled to about 4° C. After crystal formation was observed in each solution, the solutions were allowed to warm to room temperature (about 20° C.). Each solution was observed at about 20° C. over a period of at least 14 days to determine if the crystals would re-dissolve. The absence of crystals after 14 days indicates effective dissolution of tinidazole and terconazole. Table 3 includes dissolution data for a number of compositions comprising varying amounts of tinidazole, terconazole and citric acid. The data in Table 3 clearly indicate that solutions having a tinidazole concentration of at least about 0.6 percent by weight and a terconazole concentration of about 0.8 percent by weight and free from crystals at 20° C. were obtained utilizing citric acid.

TABLE 3

| | Presence or |
|---|---|
| | Absence of |
| | Crystals after |
| | at least 14 days |
| Formulation | at about 20° C. |
| Tinidazole (0.45%) + Terconazole (0.8%) + citric acid | no crystals |
| Tinidazole (0.5%) + Terconazole (0.8%) + citric acid | no crystals |
| Tinidazole (0.55%) + Terconazole (0.8%) + citric acid | no crystals |
| Tinidazole (0.6%) + Terconazole (0.8%) + citric acid | no crystals |
| Tinidazole (0.7%) + Terconazole (0.8%) + citric acid | crystals |

Surprisingly, when a colorless aqueous solution of tinidazole and citric acid was added to a colorless solution of terconazole and citric acid, a yellow color was observed. While not wishing to be bound by theory, it is believed that a complex forms between tinidazole and terconazole, perhaps also including the citric acid. As discussed below, the combination of tinidazole and terconazole also exhibits unexpected dual-drug cooperation in the inhibition of certain bacterial species, which may be associated with the complex formation.

### EXAMPLE 4

### Inhibition of Bacteria Growth by Tinidazole and Terconazole

Serial dilutions of tinidazole and terconazole were prepared by adding measured amounts of each substance to water (both individually and in combination). Seven bacterial organisms that are common pathogens in bacterial vaginosis were grown anaerobically in the medium as listed in Table 4 under a N₂/H₂/CO₂ (85:5:10) head space atmosphere.

TABLE 4

| Organism | Media |
|---|---|
| Bacillus fragilis ATCC 25285 | Difco anaerobic medium (DAMM) |
| Mobiluncus curtisii var. curtisii ATCC 35241 | Difco anaerobic medium (DAMM) + Starch (1%) + Rabbit |
| | Serum (2%) |
| Mobiluncus curtisii var. holmesii ATCC 35242 | Difco anaerobic medium (DAMM) + Starch (1%) + Rabbit |
| | Serum (2%) |
| Mobiluncus mulieris ATCC 35243 | Difco anaerobic medium (DAMM) + Starch (1%) + Rabbit |
| | Serum (2%) |
| Gardnerella vaginalis ATCC 14018 | Difco anaerobic medium (DAMM) + Rabbit |
| | Serum (2%) |
| Gardnerella vaginalis ATCC 14019 | Difco anaerobic medium (DAMM) + Rabbit |
| | Serum (2%) |
| Atopobium vaginae BAA-55 | Difco anaerobic medium (DAMM) + Vitamin K |
| | (1 ug/ml) + Hemin (5 μg/ml) + Laked Horse |
| | Blood (5%) |

These cultures were diluted to a 0.5 McFarland standard prior to inoculation of drug dilution tubes. One (1) mL of the 0.5 McFarland preparation was inoculated to an anaerobically prepared test tube containing 0.8 mL of Difco anaerobic medium (DAMM) and 0.2 mL of appropriate diluted antimicrobial or antimicrobial combination for a total of 2 mL. The inoculated tubes were then incubated at 37° C. and monitored daily for growth. Growth was scored with a (+) for growth, (−) for no growth, and (±) for suboptimal growth after an initial observation of growth. The results for each of the seven bacterial organisms are listed in Tables 5 through 11 below. Tinidazole is abbreviated as TNZ and terconazole is abbreviated as TER in Tables 5 through 11.

TABLE 5
Bacteroides fragilis ATCC 25285

| | DAY | DAY | | | |
|---|---|---|---|---|---|
| | 1 | 2 | DAY 3 | DAY 4 | DAY 5 |
| TNZ 500 μg/ml | (−) | (−) | (−) | (−) | (−) |
| TNZ 250 μg/ml | (−) | (−) | (−) | (−) | (−) |
| TNZ 125 μg/ml | (−) | (−) | (−) | (−) | (−) |
| TNZ 62.5 μg/ml | (−) | (−) | (−) | (−) | (−) |
| TNZ 31.3 μg/ml | (−) | (−) | (−) | (−) | (−) |
| TNZ 15.6 μg/ml | (−) | (−) | (−) | (−) | (−) |
| TNZ 7.8 μg/ml | (−) | (−) | (−) | (−) | (−) |
| TNZ 3.9 μg/ml | (−) | (−) | (−) | (−) | (−) |
| TNZ 2 μg/ml | (−) | (−) | (−) | (−) | (+) |
| TER 800 μg/ml | (−) | (−) | (−) | (−) | (−) |
| TER 400 μg/ml | (−) | (−) | (−) | (−) | (−) |
| TER 200 μg/ml | (−) | (−) | (+) | (+) | (+) |
| TER 100 μg/ml | (+) | (+) | (+) | (+) | (+) |
| TER 50 μg/ml | (+) | (+) | (+) | (+) | (+) |
| TER 25 μg/ml | (+) | (+) | (+) | (+) | (+) |
| TER 12.5 μg/ml | (+) | (+) | (+) | (+) | (+) |
| TER 6.2 μg/ml | (+) | (+) | (+) | (+) | (+) |
| TER 3.1 μg/ml | (+) | (+) | (+) | (+) | (+) |
| TNZ 500 + TER 800 μg/ml | (−) | (−) | (−) | (−) | (−) |
| TNZ 250 + TER 400 μg/ml | (−) | (−) | (−) | (−) | (−) |
| TNZ 125 + TER 200 μg/ml | (−) | (−) | (−) | (−) | (−) |
| TNZ 62.5 + TER 100 μg/ml | (−) | (−) | (−) | (−) | (−) |
| TNZ 31.3 + TER 50 μg/ml | (−) | (−) | (−) | (−) | (−) |
| TNZ 15.6 + TER 25 μg/ml | (−) | (−) | (−) | (−) | (−) |
| TNZ 7.8 + TER 12.5 μg/ml | (−) | (−) | (−) | (−) | (−) |
| TNZ 3.9 + TER 6.2 μg/ml | (−) | (−) | (−) | (−) | (−) |
| TNZ 2 + TER 3.1 μg/ml | (−) | (−) | (−) | (−) | (+) |

TABLE 6
Mobiluncus curtisii var. curtisii ATCC 35241

| | DAY | DAY | | | |
|---|---|---|---|---|---|
| | 1 | 2 | DAY 3 | DAY 4 | DAY 5 |
| TNZ 500 μg/ml | (−) | (−) | (−) | (−) | (+) |
| TNZ 250 μg/ml | (−) | (+) | (+) | (+) | (+) |
| TNZ 125 μg/ml | (+) | (+) | (+) | (+) | (+) |
| TNZ 62.5 μg/ml | (+) | (+) | (+) | (+) | (+) |
| TNZ 31.3 μg/ml | (+) | (+) | (+) | (+) | (+) |
| TNZ 15.6 μg/ml | (+) | (+) | (+) | (+) | (+) |
| TNZ 7.8 μg/ml | (+) | (+) | (+) | (+) | (+) |
| TNZ 3.9 μg/ml | (+) | (+) | (+) | (+) | (+) |
| TNZ 2 μg/ml | (+) | (+) | (+) | (+) | (+) |
| TER 800 μg/ml | (−) | (−) | (−) | (−) | (+) |
| TER 400 μg/ml | (−) | (−) | (−) | (−) | (+) |
| TER 200 μg/ml | (−) | (+) | +/− | +/− | (+) |
| TER 100 μg/ml | (+) | (+) | (+) | (+) | (+) |
| TER 50 μg/ml | (+) | (+) | (+) | (+) | (+) |
| TER 25 μg/ml | (+) | (+) | (+) | (+) | (+) |
| TER 12.5 μg/ml | (+) | (+) | (+) | (+) | (+) |
| TER 6.2 μg/ml | (+) | (+) | (+) | (+) | (+) |
| TER 3.1 μg/ml | (+) | (+) | (+) | (+) | (+) |
| TNZ 500 + TER 800 μg/ml | (−) | (−) | (−) | (−) | (−) |
| TNZ 250 + TER 400 μg/ml | (−) | (−) | (−) | (−) | (−) |
| TNZ 125 + TER 200 μg/ml | (−) | (−) | (−) | (−) | (−) |
| TNZ 62.5 + TER 100 μg/ml | (+) | (+) | (+) | (+) | (+) |
| TNZ 31.3 + TER 50 μg/ml | (+) | (+) | (+) | (+) | (+) |
| TNZ 15.6 + TER 25 μg/ml | (+) | (+) | (+) | (+) | (+) |
| TNZ 7.8 + TER 12.5 μg/ml | (+) | (+) | (+) | (+) | (+) |
| TNZ 3.9 + TER 6.2 μg/ml | (+) | (+) | (+) | (+) | (+) |
| TNZ 2 + TER 3.1 μg/ml | (+) | (+) | (+) | (+) | (+) |

TABLE 7
Mobiluncus curtisii var. holmesii ATCC 35242

| | DAY | DAY | | | |
|---|---|---|---|---|---|
| | 1 | 2 | DAY 3 | DAY 4 | DAY 5 |
| TNZ 500 μg/ml | (+) | (+) | (+) | (+) | (+) |
| TNZ 250 μg/ml | (+) | (+) | (+) | (+) | (+) |
| TNZ 125 μg/ml | (+) | (+) | (+) | (+) | (+) |
| TNZ 62.5 μg/ml | (+) | (+) | (+) | (+) | (+) |
| TNZ 31.3 μg/ml | (+) | (+) | (+) | (+) | (+) |
| TNZ 15.6 μg/ml | (+) | (+) | (+) | (+) | (+) |
| TNZ 7.8 μg/ml | (+) | (+) | (+) | (+) | (+) |
| TNZ 3.9 μg/ml | (+) | (+) | (+) | (+) | (+) |
| TNZ 2 μg/ml | (+) | (+) | (+) | (+) | (+) |
| TER 800 μg/ml | (+) | +/− | +/− | +/− | (+) |
| TER 400 μg/ml | (+) | +/− | +/− | +/− | (+) |
| TER 200 μg/ml | (+) | +/− | +/− | +/− | (+) |
| TER 100 μg/ml | (+) | (+) | (+) | (+) | (+) |
| TER 50 μg/ml | (+) | (+) | (+) | (+) | (+) |
| TER 25 μg/ml | (+) | (+) | (+) | (+) | (+) |
| TER 12.5 μg/ml | (+) | (+) | (+) | (+) | (+) |
| TER 6.2 μg/ml | (+) | (+) | (+) | (+) | (+) |
| TER 3.1 μg/ml | (+) | (+) | (+) | (+) | (+) |
| TNZ 500 + TER 800 μg/ml | (+) | +/− | +/− | +/− | +/− |
| TNZ 250 + TER 400 μg/ml | (+) | +/− | +/− | +/− | +/− |
| TNZ 125 + TER 200 μg/ml | (+) | +/− | +/− | +/− | +/− |
| TNZ 62.5 + TER 100 μg/ml | (+) | +/− | +/− | +/− | (+) |
| TNZ 31.3 + TER 50 μg/ml | (+) | (+) | (+) | (+) | (+) |
| TNZ 15.6 + TER 25 μg/ml | (+) | (+) | (+) | (+) | (+) |
| TNZ 7.8 + TER 12.5 μg/ml | (+) | (+) | (+) | (+) | (+) |
| TNZ 3.9 + TER 6.2 μg/ml | (+) | (+) | (+) | (+) | (+) |
| TNZ 2 + TER 3.1 μg/ml | (+) | (+) | (+) | (+) | (+) |

TABLE 8
Mobiluncus mulieris ATCC 35243

| | DAY | DAY | | | |
|---|---|---|---|---|---|
| | 1 | 2 | DAY 3 | DAY 4 | DAY 5 |
| TNZ 500 μg/ml | (−) | (−) | (−) | (−) | (−) |
| TNZ 250 μg/ml | (−) | (−) | (−) | (−) | (−) |
| TNZ 125 μg/ml | (−) | (−) | (−) | (−) | (−) |
| TNZ 62.5 μg/ml | (−) | (−) | (−) | (−) | (−) |
| TNZ 31.3 μg/ml | (−) | (−) | (−) | (+) | (+) |
| TNZ 15.6 μg/ml | (−) | (−) | (−) | (+) | (+) |
| TNZ 7.8 μg/ml | (+) | (+) | (+) | (+) | (+) |
| TNZ 3.9 μg/ml | (+) | (+) | (+) | (+) | (+) |
| TNZ 2 μg/ml | (+) | (+) | (+) | (+) | (+) |
| TER 800 μg/ml | (−) | (−) | (−) | (−) | (−) |
| TER 400 μg/ml | (−) | (−) | (−) | (−) | (−) |
| TER 200 μg/ml | (+) | (+) | (+) | (+) | (+) |
| TER 100 μg/ml | (+) | (+) | (+) | (+) | (+) |
| TER 50 μg/ml | (+) | (+) | (+) | (+) | (+) |
| TER 25 μg/ml | (+) | (+) | (+) | (+) | (+) |
| TER 12.5 μg/ml | (+) | (+) | (+) | (+) | (+) |
| TER 6.2 μg/ml | (+) | (+) | (+) | (+) | (+) |
| TER 3.1 μg/ml | (+) | (+) | (+) | (+) | (+) |
| TNZ 500 + TER 800 μg/ml | (−) | (−) | (−) | (−) | (−) |
| TNZ 250 + TER 400 μg/ml | (−) | (−) | (−) | (−) | (−) |
| TNZ 125 + TER 200 μg/ml | (−) | (−) | (−) | (−) | (−) |
| TNZ 62.5 + TER 100 μg/ml | (−) | (−) | (−) | (−) | (−) |
| TNZ 31.3 + TER 50 μg/ml | (−) | (−) | (−) | (−) | (+) |
| TNZ 15.6 + TER 25 μg/ml | (−) | (−) | (−) | (−) | (+) |
| TNZ 7.8 + TER 12.5 μg/ml | (+) | (+) | (+) | (+) | (+) |
| TNZ 3.9 + TER 6.2 μg/ml | (+) | (+) | (+) | (+) | (+) |
| TNZ 2 + TER 3.1 μg/ml | (+) | (+) | (+) | (+) | (+) |

TABLE 9
Gardnerella vaginalis ATCC 14018

| | DAY | DAY | | | |
|---|---|---|---|---|---|
| | 1 | 2 | DAY 3 | DAY 4 | DAY 5 |
| TNZ 500 μg/ml | (−) | (−) | (−) | (−) | (−) |
| TNZ 250 μg/ml | (−) | (−) | (−) | (−) | (−) |
| TNZ 125 μg/ml | (−) | (−) | (−) | (−) | (−) |
| TNZ 62.5 μg/ml | (−) | (−) | (−) | (−) | (−) |
| TNZ 31.3 μg/ml | (−) | (−) | (−) | (−) | (−) |
| TNZ 15.6 μg/ml | (−) | (+) | (+) | (+) | (+) |
| TNZ 7.8 μg/ml | (−) | (+) | (+) | (+) | (+) |
| TNZ 3.9 μg/ml | (+) | (+) | (+) | (+) | (+) |
| TNZ 2 μg/ml | (+) | (+) | (+) | (+) | (+) |
| TER 800 μg/ml | (−) | (−) | (−) | (−) | (−) |
| TER 400 μg/ml | (−) | (−) | (−) | (−) | (−) |
| TER 200 μg/ml | (−) | (+) | (+) | (+) | (+) |
| TER 100 μg/ml | (+) | (+) | (+) | (+) | (+) |
| TER 50 μg/ml | (+) | (+) | (+) | (+) | (+) |
| TER 25 μg/ml | (+) | (+) | (+) | (+) | (+) |
| TER 12.5 μg/ml | (+) | (+) | (+) | (+) | (+) |
| TER 6.2 μg/ml | (+) | (+) | (+) | (+) | (+) |
| TER 3.1 μg/ml | (+) | (+) | (+) | (+) | (+) |
| TNZ 500 + TER 800 μg/ml | (−) | (−) | (−) | (−) | (−) |
| TNZ 250 + TER 400 μg/ml | (−) | (−) | (−) | (−) | (−) |
| TNZ 125 + TER 200 μg/ml | (−) | (−) | (−) | (−) | (−) |
| TNZ 62.5 + TER 100 μg/ml | (−) | (−) | (−) | (−) | (−) |
| TNZ 31.3 + TER 50 μg/ml | (−) | (−) | (−) | (−) | (−) |
| TNZ 15.6 + TER 25 μg/ml | (−) | (−) | (−) | (+) | (+) |
| TNZ 7.8 + TER 12.5 μg/ml | (−) | (+) | (+) | (+) | (+) |
| TNZ 3.9 + TER 6.2 μg/ml | (+) | (+) | (+) | (+) | (+) |
| TNZ 2 + TER 3.1 μg/ml | (+) | (+) | (+) | (+) | (+) |

TABLE 10
Gardnerella vaginalis ATCC 14019

| | DAY | DAY | | | |
|---|---|---|---|---|---|
| | 1 | 2 | DAY 3 | DAY 4 | DAY 5 |
| TNZ 500 μg/ml | (+) | (+) | (+) | (+) | (+) |
| TNZ 250 μg/ml | (+) | (+) | (+) | (+) | (+) |
| TNZ 125 μg/ml | (+) | (+) | (+) | (+) | (+) |
| TNZ 62.5 μg/ml | (+) | (+) | (+) | (+) | (+) |
| TNZ 31.3 μg/ml | (+) | (+) | (+) | (+) | (+) |
| TNZ 15.6 μg/ml | (+) | (+) | (+) | (+) | (+) |
| TNZ 7.8 μg/ml | (+) | (+) | (+) | (+) | (+) |
| TNZ 3.9 μg/ml | (+) | (+) | (+) | (+) | (+) |
| TNZ 2 μg/ml | (+) | (+) | (+) | (+) | (+) |
| TER 800 μg/ml | (−) | (−) | (−) | (−) | (−) |
| TER 400 μg/ml | (+) | (+) | (+) | (+) | (+) |
| TER 200 μg/ml | (+) | (+) | (+) | (+) | (+) |
| TER 100 μg/ml | (+) | (+) | (+) | (+) | (+) |
| TER 50 μg/ml | (+) | (+) | (+) | (+) | (+) |
| TER 25 μg/ml | (+) | (+) | (+) | (+) | (+) |
| TER 12.5 μg/ml | (+) | (+) | (+) | (+) | (+) |
| TER 6.2 μg/ml | (+) | (+) | (+) | (+) | (+) |
| TER 3.1 μg/ml | (+) | (+) | (+) | (+) | (+) |
| TNZ 500 + TER 800 μg/ml | (−) | (−) | (−) | (−) | (−) |
| TNZ 250 + TER 400 μg/ml | (+) | (+) | (+) | (+) | (+) |
| TNZ 125 + TER 200 μg/ml | (+) | (+) | (+) | (+) | (+) |
| TNZ 62.5 + TER 100 μg/ml | (+) | (+) | (+) | (+) | (+) |
| TNZ 31.3 + TER 50 μg/ml | (+) | (+) | (+) | (+) | (+) |
| TNZ 15.6 + TER 25 μg/ml | (+) | (+) | (+) | (+) | (+) |
| TNZ 7.8 + TER 12.5 μg/ml | (+) | (+) | (+) | (+) | (+) |
| TNZ 3.9 + TER 6.2 μg/ml | (+) | (+) | (+) | (+) | (+) |
| TNZ 2 + TER 3.1 μg/ml | (+) | (+) | (+) | (+) | (+) |

TABLE 11
Atopobium vaginae BAA-55

| | DAY | DAY | | | |
|---|---|---|---|---|---|
| | 1 | 2 | DAY 3 | DAY 4 | DAY 5 |
| TNZ 500 μg/ml | (−) | (−) | (+) | (+) | (+) |
| TNZ 250 μg/ml | (−) | (+) | (+) | (+) | (+) |
| TNZ 125 μg/ml | (−) | (+) | (+) | (+) | (+) |
| TNZ 62.5 μg/ml | (−) | (+) | (+) | (+) | (+) |
| TNZ 31.3 μg/ml | (−) | (+) | (+) | (+) | (+) |
| TNZ 15.6 μg/ml | (−) | (+) | (+) | (+) | (+) |
| TNZ 7.8 μg/ml | (+) | (+) | (+) | (+) | (+) |
| TNZ 3.9 μg/ml | (+) | (+) | (+) | (+) | (+) |
| TNZ 2 μg/ml | (+) | (+) | (+) | (+) | (+) |
| TER 800 μg/ml | (−) | (−) | (−) | (−) | (−) |
| TER 400 μg/ml | (−) | (−) | (−) | (−) | (−) |
| TER 200 μg/ml | (−) | (−) | (−) | (+) | (+) |
| TER 100 μg/ml | (−) | (−) | (+) | (+) | (+) |
| TER 50 μg/ml | (+) | (+) | (+) | (+) | (+) |
| TER 25 μg/ml | (+) | (+) | (+) | (+) | (+) |
| TER 12.5 μg/ml | (+) | (+) | (+) | (+) | (+) |
| TER 6.2 μg/ml | (+) | (+) | (+) | (+) | (+) |
| TER 3.1 μg/ml | (+) | (+) | (+) | (+) | (+) |
| TNZ 500 + TER 800 μg/ml | (−) | (−) | (−) | (−) | (−) |
| TNZ 250 + TER 400 μg/ml | (−) | (−) | (−) | (−) | (−) |
| TNZ 125 + TER 200 μg/ml | (−) | (−) | (−) | (−) | (−) |
| TNZ 62.5 + TER 100 μg/ml | (−) | (−) | (+) | (+) | (+) |
| TNZ 31.3 + TER 50 μg/ml | (−) | (+) | (+) | (+) | (+) |
| TNZ 15.6 + TER 25 μg/ml | (−) | (+) | (+) | (+) | (+) |
| TNZ 7.8 + TER 12.5 μg/ml | (−) | (+) | (+) | (+) | (+) |
| TNZ 3.9 + TER 6.2 μg/ml | (+) | (+) | (+) | (+) | (+) |
| TNZ 2 + TER 3.1 μg/ml | (+) | (+) | (+) | (+) | (+) |

The data in Tables 6 through 11 indicate that the combination of tinidazole and terconazole unexpectedly exhibits a non-additive dual-drug cooperation in the inhibition of Mobiluncus curtisii var. curtisii, as well as var. holmesii and Atopobium vaginae. In addition, terconazole, which is generally used for yeast infections, unexpectedly exhibited antibacterial activity against Mobiluncus mulieris, Gardnerella vaginalis and Atopobium vaginae, which was superior to the antibacterial compound tinidazole.

### EXAMPLE 5

### A Tinidazole/Terconazole Gel Composition

A gel composition containing the ingredients listed in Table 12 was prepared, which comprised about 0.5 percent by weight tinidazole and 0.8 percent by weight of terconazole.

TABLE 12

| Ingredient | Percentage |
|---|---|
| Terconazole | 0.8 |
| Tinidazole | 0.50 |
| MethoCel K100M | 2 |
| Propylene glycol | 5 |
| Methyl paraben | 0.08 |
| Propyl paraben | 0.02 |
| Citric acid | 0.3 |
| Sodium citrate | 0.15 |
| EDTA | 0.05 |
| Water | Q.S. to 100% |

The gel composition was free from tinidazole and terconazole crystals.

Numerous variations and modifications of the embodiments described above may be effected without departing from the spirit and scope of the novel features of the invention. No limitations with respect to the specific embodiments illustrated herein are intended or should be inferred.

We claim:

1. An aqueous composition comprising at least about 0.5 percent by weight tinidazole dissolved in water; at least about 0.4 percent by weight of terconazole dissolved in water; and a crystallization-inhibiting amount of an organic acid;
the composition being free from terconazole and tinidazole crystals at an ambient temperature of about 20° C.

2. The composition of claim 1 wherein the solution comprises up to about 0.6 percent by weight of tinidazole.

3. The composition of claim 1 wherein the solution comprises at least about 0.8 percent by weight of terconazole.

4. The composition of claim 1 wherein the solution comprises up to about 3 percent by weight of terconazole.

5. The composition of claim 1 wherein the organic acid comprises citric acid.

6. The composition of claim 1 wherein the organic acid is present in the composition in a molar amount at least equal to the combined molar amount of tinidazole and terconazole dissolved in the composition.

7. The composition of claim 1 wherein the organic acid is present in the composition at a concentration in the range of about 50 to about 150 mole percent based upon the combined molar amount of tinidazole and terconazole dissolved in the composition.

8. The composition of claim 1 further comprising a thickening agent.

9. The composition of claim 8 wherein the thickening agent comprises a hydroxypropyl methylcellulose.

10. An aqueous composition comprising at least about 0.8 percent by weight of terconazole dissolved in water; about 0.5 to about 0.6 percent by weight tinidazole dissolved in water; and a crystallization-inhibiting amount of citric acid;
the composition being free from terconazole and tinidazole crystals at an ambient temperature of about 20° C.

11. The composition of claim 10 wherein the citric acid is present in the composition in a molar amount at least equal to the combined molar amount of tinidazole and terconazole dissolved in the composition.

12. The composition of claim 10 wherein the citric acid is present in 5 the composition at a concentration in the range of about 50 to about 150 mole percent based upon the combined molar amount of tinidazole and terconazole dissolved in the composition.

13. The composition of claim 10 further comprising a thickening agent.

14. The composition of claim 13 wherein the thickening agent comprises a hydroxypropyl methylcellulose.

15. A method for preparing a solution comprising at least about 0.5 percent by weight of tinidazole dissolved in water and at least about 0.4 percent by weight of terconazole dissolved in water, free from terconazole and tinidazole crystals at an ambient temperature of about 20° C., the method comprising dissolving terconazole and tinidazole in an aqueous solution containing a crystallization-inhibiting amount of an organic acid.

16. The method of claim 15 wherein the solution comprises at least about 0.8 percent by weight of terconazole.

17. The method of claim 15 wherein the solution comprises up to about 3 percent by weight of terconazole.

18. The method of claim 15 wherein the solution comprises up to about 0.6 percent by weight of tinidazole.

19. The method of claim 15 wherein the organic acid is present in the solution in a molar amount at least equal to the combined molar amount of tinidazole and terconazole dissolved in the composition.

20. The method of claim 15 wherein the organic acid is present in the solution at a concentration in a range of about 50 to about 150 mole percent based on the combined molar amount of tinidazole and terconazole dissolved in the composition.

21. The method of claim 15 wherein the organic acid comprises citric acid.

22. An article of manufacture for treating fungal infections comprising packaging material and at least one composition of claim 1 in at least one sealed container within the packaging material; the at least one container bearing a label that includes written indicia describing the contents thereof.
